(19) United States
(12) Plant Patent Application Publication
(10) Pub. No.: US 2021/0204458 P1
(43) Pub. Date: Jul. 1, 2021

(54) SYCAMORE PLANT NAMED DRABROT

(50) ;

(71) Applicants: Timothy C. Brotzman, Madison, OH (US); Thomas M. Draves, Darien, NY (US)

(72) Inventors: Timothy C. Brotzman, Madison, OH (US); Thomas M. Draves, Darien, NY (US)

(21) Appl. No.: 17/135,807

(22) Filed: Dec. 28, 2020

Related U.S. Application Data
(60) Provisional application No. 62/974,918, filed on Dec. 31, 2019.

(51) Int. Cl. A01H 6/00

(52) U.S. Cl. PLT/216

(57) ABSTRACT

A new variety of Platanus occidentalis named DRABROT which is characterized by a contorted, semi-weeping branch habit and dark green, disease-free leaves. In combination, these characteristics distinguish DRABROT from all other cultivars known to the inventors.

Genus: Platanus.

Species: occidentalis.

Denomination: DRABROT.

### BACKGROUND OF THE INVENTION

This application claims the benefit of priority under U.S.C. 119(e) of U.S. Provisional Application No. 62/974,918 filed on Dec. 31, 2019 entitled SYCAMORE PLANT NAMED ‘DRABROT’ and for which the inventors now wish to file this application with the name or denomination DRABROT.

The present invention relates to a new and distinct cultivar of Platanus occidentalis (American Sycamore or American Planetree) which is grown as a large ornamental tree for use in parks and landscape. The new cultivar is known botanically as Platanus occidentalis and will be referred to hereinafter by the cultivar name DRABROT.

The DRABROT variety was found by Thomas M. Draves, ISA Certified Arborist, as a specimen tree incorporated into the landscape of a residence in Darien, N.Y. and was recognized by him as unique for its contorted habit and semi-weeping branches.

The DRABROT variety is the only variant of American Sycamore we are aware of that displays this form when compared to the following commercial selections: Platanus occidentalis ‘Howard’ U.S. Plant Pat. No. 5,359 with an arboreal form typical of the species but slower growth and yellow leaves; Platanus occidentalis ‘Grenickle’ (not patented) SILVERWOOD™, height and width 75-100′, bark white. The majority of commercially produced Platanus are forms of the hybrid Platanus x acerifolia (Platanus occidentalis x Platanus orientalis, commonly known as London Planetree, none of which have the unique form of the DRABROT selection, and as noted in The Tree Book, by Din & Warren, Timber Press 2019, are represented by the following cultivars: Platanus x acerifolia ‘Bloodgood’ (70′ tall×50′ wide); Platanus x acerifolia ‘Columbia’ (70′ tall×50′ wide, USDA hardiness zone 6), Platanus x acerifolia ‘Morton Circle’ (Exclamation™) (60′ tall×30′ wide); and Platanus x acerifolia ‘Suttneri’ (60′ tall×45′ wide, bark almost pure white).

The original tree is estimated at over 60 years of age with an approximate size of 35′ tall by 48′ wide, and two trunks with diameters of 10″ and 12″. A 17-year old single-stein specimen in the Draves Arboretum measures 16′ tall×21′ wide, and has a diameter of 6″ at a point 6″ above the ground.

### ASEXUAL PROPAGATION OF THE INVENTION

DRABROT was first asexually propagated by Tim Brotzman, co-inventor, in summer of 2003 in Madison, Ohio. Asexual propagation has been accomplished using softwood cuttings and by budding onto rootstock of Platanus occidentalis. Since that time, under careful observation, the distinguishing characteristics of DRABROT have been determined stable and uniform, and to reproduce true to type in successive generations of asexual propagation.

### SPECIFICATION DESCRIPTION OF THE INVENTION

Using observations collected from 17-year-old trees growing at Brotzman's Nursery, Inc, Madison, Ohio and at the Draves Arboretum, Darien, N.Y., as well as the mother tree, we have identified traits which in combination set DRABROT apart from all other existing varieties of Platanus known to the inventors. DRABROT has not been tested under all possible conditions and phenotypic differences may be observed with variations in environmental, climatic, and cultural conditions. The distinguishing characteristics of DRABROT are as follows:

1. DRABROT exhibits a contorted, semi-weeping branch habit.

2. DRABROT lacks strong apical dominance

3. The leaves of DRABROT are dark green.

4. The leaves of DRABROT have shown high degree of resistance to Leaf Anthracnose (Apiognomonia veneta) and Powdery Mildew (Erysiphe platani) in both Madison, Ohio and Darien, N.Y.

5. When grown as a single stein tree DRABROT probably has a mature height and width of less than 35′×40′.

6. The mature trunk of DRABROT has exfoliating bark, revealing a broken pattern of green-grayed-white colored patches.

7. DRABROT is hardy at least to USDA Zone 5B.

### BOTANICAL DESCRIPTION

Note: all references to color are using the R.H.S. Colour Chart, published by The Royal Horticultural Society, London, England 1966
Parentage: Originated as a spontaneous seedling of American Sycamore, Platanus occidentalis.
Hardiness: USDA Hardiness Zone 5b
Growth rate: Slow compared to the species and hybrids. When propagated by chip budding, 1-yr whips of DRABROT averaged 44.2″ in height, compared to Platanus x acerifolia ‘Morton Circle’ which averaged 72.4″ at the same age. Three-year old DRABROT exhibited the following averages: 27″ annual lateral growth, 33″ vertical growth, overall total height of 114″ and diameter of 1.35″ when measured 6″ above the ground, compared to three-year-old Platanus occidentalis with averages of 36″ lateral growth , 72″ vertical growth, overall height of 192″ and diameter of 2.55″ under field conditions in Madison, Ohio.
Form/habit: From a short central stein, rather than producing a central leader, multiple primary branches arch outward radially at angles of 35° to 70° (degrees) from vertical. As these branches elongate, they contort and the tips may flatten horizontally or droop as much as 120° to 160° from vertical, yielding an umbrella-shaped crown with semi-weeping habit.
Bark: The bark on the trunk of young trees is tight and dark, (Greyed-Green Group 195-A), with age developing lighter colored exfoliating plates (Greyed-Green Group 195-C to Greyed-Green Group 195-A) until finally on old plants nearly all white (White Group 155-C).
Leaves: Arranged alternately on stems, with 3-5 lobes and toothed margins. Leaf blade to 8 inches or more wide and 6 inches tall. Leaf color dark green (Green Group 135-C). Upper surface of leaf smooth, underside slightly hairy, especially on the veins.
Twigs/buds: The conical buds are dark, (Greyed-Orange Group 166-A). During the growing season, the buds are hidden within the swollen, hollow base of the leaf petiole, completely enclosing it. New growth (twigs) Grey-Orange Group 165-A, darkening on larger (1″ diameter) branches (Grey-Green Group 197-B to Greyed-Green Group-A).
Flowers/fruit: As for the species, male and female flowers, not showy, are borne in separate, drooping, ball-like heads. The female heads of flowers develop into 1″ diameter globose heads of seeds. The styles from the flowers extend from the ends of the seeds in the fruits, but bristles, such as are present in the fruits of London Planetree, are absent. Fruit color is Grey-Orange Group 164-C.

### DESCRIPTION OF THE DRAWINGS

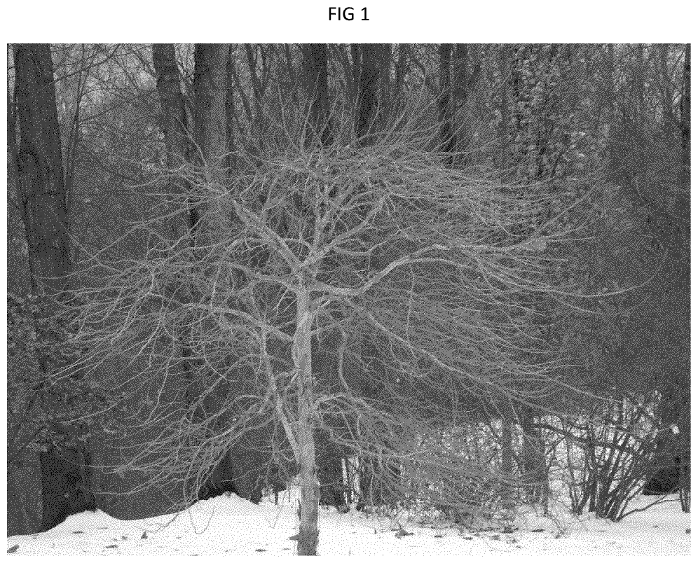

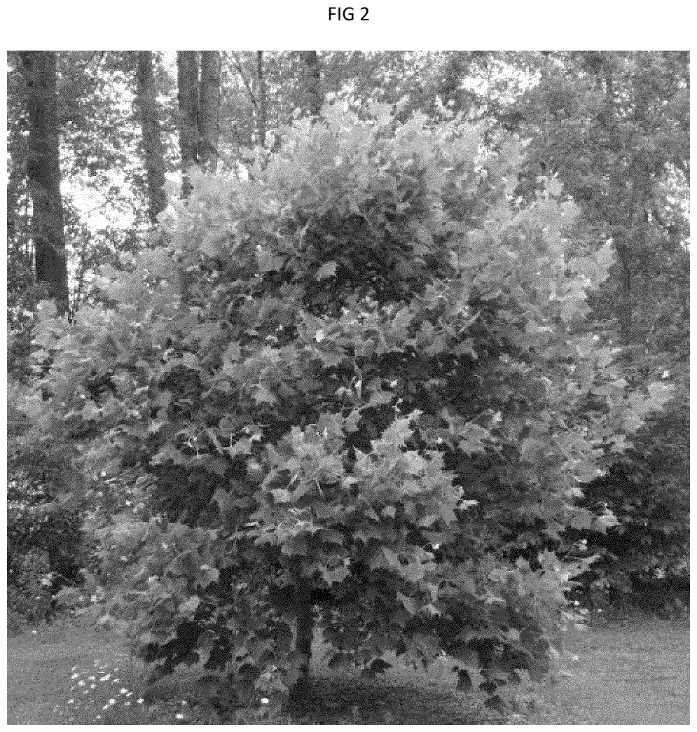

The accompanying color drawing, labeled FIG. 1, illustrates the overall appearance of DRABROT showing the semi-weeping and contorted growth habit, and the exfoliating bark patches, as exhibited on a 17-year old dormant specimen in a landscape application.

1. A new and distinct variety of Platanus occidentalis named DRABROT, substantially as described and illustrated herein.
